(19) United States
(12) Patent Application Publication
(10) Pub. No.: US 2009/0186101 A1
(43) Pub. Date: Jul. 23, 2009

(54) Use of Elderberry Extract

(75) Inventor: Madeleine Mumcuoglu, Jerusalem (IL)

Correspondence Address: FITZPATRICK CELLA HARPER & SCINTO, 30 ROCKEFELLER PLAZA, NEW YORK, NY, 10112, US

(73) Assignee: HEALTHCARE BRANDS INTERNATIONAL LIMITED, LONDON (GB)

(21) Appl. No.: 12/159,691

(22) PCT Filed: Jan. 17, 2007
(86) PCT No.: PCT/GB2007/000150

§ 371 Date: Dec. 5, 2008

(30) Foreign Application Priority Data
Jan. 17, 2006 (IL) 173207

(51) Int. Cl. A61K 36/00; A61P 31/12; C12N 5/06

(52) U.S. Cl. 424/725; 435/375

(57) ABSTRACT

The present invention relates to the field of viral therapy. More specifically, the present invention relates to the use of elderberry extract in the preparation of a pharmaceutical formulation for the treatment of an influenza viral infection. It also relates to a novel method of treatment of the avian flu virus, through the administration of elderberry extract.

The present invention relates to the field of viral therapy. More specifically, the present invention relates to the use of elderberry extract in the preparation of a pharmaceutical formulation for the treatment of an influenza viral infection. It also relates to a novel method of treatment of the avian flu virus, through the administration of elderberry extract.

All publications mentioned throughout this application are fully incorporated herein by reference, including all references cited therein.

The spread of avian influenza from the Far East to East European Countries raises concerns of a possible flu pandemic and emphasises the need to search for a preventative and/or cure.

Influenza viruses belong to a broad family of RNA viruses, the Orthomyxoviridae (myxo=mucus) viruses. Orthomyxoviridae viruses as well as the closely related family of RNA viruses, Paramyxoviridae viruses, are characterised by a negative-stranded RNA genome (segmented or non-segmented, respectively), having an inner ribonucleoprotein (RNP) core surrounded by a lipid bilayer membrane from which spikes protrude. The spikes are of three kinds: a haemagglutinin (HA) which agglutinates erythrocytes, an enzyme neuraminidase (NA) which releases the virus from cells, and a small number of copies of the M2 protein that serves as an ion channel. These spikes in influenza (in Paramyxoviridae virus these are HN and F) are involved in haemagglutination, haemolysis of erythrocytes etc. and cleavage of the receptor (on the cell) anti-receptor (on the virus) bond, and reflect the ability of the virus to enter the nucleoprotein core into cells.

The past century has witnessed three pandemics of influenza, of which the “Spanish flu” of 1918 was the largest. The World Health Organisation (WHO) and experts around the world claim that a new influenza pandemic will occur in the near future. The current global concern is the avian influenza A (H5N1) virus, which first demonstrated its ability to infect birds in China in 1997 and has since spread to other countries in South East Asia and also, via migrating birds, the East European countries. According to the WHO, the year 2005 ended with a total of 141 confirmed human cases of avian influenza A (H5N1) of which 73 were fatal [http://www.who.intlcsr/disease/avian_influenza/en/index.html].

The designation H5N1 is in accordance with the convention for naming influenza viruses. The H and N stand for haemagglutinin and neuraminidase, respectively. Haemagglutinin is an antigenic glycoprotein found on the surface of the influenza virus, which binds the virus to the host cell being infected. Neuraminidase is a glycoside hydrolase enzyme which is also found on the surface of the influenza virus.

Influenza viruses can change in two different ways, known as “antigenic drift” and “antigenic shift”. Antigenic drift are small changes in the virus that happen continually over time, producing new virus strains that may not be recognised by the body's immune system. This is one of the main reasons why people can get the flu more than one time. IN most years, one or two of the three virus strains in the influenza vaccine are updated to keep up with the changes in the circulating flu viruses. So, people who want to be protected from flu need to get a flu shot every year. Antigenic shift is an abrupt, major change in the influenza A viruses, resulting in new haemagglutinin and/or new haemagglutinin and neuraminidase proteins in the influenza viruses that infect humans. Shift results in a new influenza A subtype. When shift happens, most people have little or no protection against the new virus. While influenza viruses are changing by antigenic drift all the time, antigenic shift happens only occasionally. Type A viruses undergo both kinds of changes; influenza type B viruses change only by the more gradual process of antigenic drift.

Thus, despite previous reports that Elderberry extract (or Sambucol®) was effective against influenza virus infection [U.S. Pat. No. 4,742,046], the present inventors set off to investigate the effectiveness of Sambucol® against avian influenza H5N1, both in vitro and in vivo. As mentioned above, the H5N1 strain of influenza type A virus is a relatively new strain of influenza virus, which is feared as the probable cause of the next flu pandemic, and for which no sufficient supplies of vaccines or antidotes are currently available.

Sambucol® is a branded herbal preparation, based on a standardised extract of the European Black Elderberry (Sambucus nigra L.). Elderberries are members of the honeysuckle family, Caprifoliaceae. American elder is classified as Sambucus canadensis, blue elder as Sambucus caerulea, and European black elder as Sambucus nigra. Purple elder is a variety of European black elder, Sambucus nigra purpurea.

The Sambucol® preparation (Razei Bar Industries, Israel) contains natural ingredients with antiviral properties [Flos Sambuci (2002) WHO monographs on selected Medicinal Plants 2:269-275]. In vitro studies with Sambucol® have shown it to reduce haemogglutination and inhibit replication of type A and type B human influenza viruses, as well as of animal strains including type A turkey influenza virus [Zakay-Rones, Z. et al (1995) Inhibition of several strains of influenza virus in vitro and reduction of symptoms by an elderberry extract (Sambucus nigra L.) during an outbreak of influenza B panama J. Altern. Complem. Med 1(4):361-369]. Furthermore, two double-blind placebo-controlled clinical trials confirmed the safety and efficacy of Sambucol® in the treatment of influenza and its symptoms. In both trials the Sambucol® group showed pronounced improvement (including overall performance and severity of symptoms). Patients receiving Sambucol® recovered 3 and 4 days, respectively, earlier than control groups, which achieved similar improvement on days 6 and 8, respectively, of the trials [Zakay-Rones (1995) ibid; Zakay-Rons, Z. (2004) Randomised study of the efficacy and safety of oral elderberry extract in the treatment of influenza A and B virus J Int Med. Res. 32(2): 132-140].

U.S. Pat. No. 4,742,046 described the activity of fractionated elderberry extract, on the virus A/PR/34 CHON1, an influenza virus type A. More specifically, the activity of Sambucus-derived lectins was tested. In the present invention, the inventors have applied a crude extract of elderberry, also referred to as Sambucol®, to cells infected with the influenza type A virus, H5N1, which has been correlated to the latest outbreaks of avian flu.

Thus it is an object of the present invention to provide the use of Sambucol®, a black elderberry extract, in the treatment of avian flu. In particular, the invention intends to provide the inhibition of viral activity and/or infectivity, through the administration of said elderberry extract to a subject in need.

In a first aspect, the present invention provides, the use of Sambucus nigra L extract in the preparation of a pharmaceutical formulation for the treatment of an influenza viral infection in a subject in need. Specifically, said influenza viral infection is caused by the influenza type A virus H5N1, and said subject in need is human. Preferably, the Sambucus nigra L extract is Sambucol®.

In a second aspect, the present invention provides a method of treating an influenza viral infection, said method comprising administering a therapeutic effective amount of a Sambucus nigra L extract to a subject in need. Specifically, said influenza viral infection is caused by the influenza type A virus H5N1, and said subject in need is a human. Preferably, the Sambucus nigra L extract is Sambucol®.

In a further aspect, the present invention provides a method of inhibiting influenza viral infection, said method comprising contacting an environment, for example cells infected with the influenza virus with an effective amount of Sambucus nigra L extract, wherein said influenza virus us the influenza type A virus H5N1. Preferably, the Sambucus nigra L extract is Sambucol®.

### BRIEF DESCRIPTION OF THE FIGURES

FIG. 1: Moderately diluted Sambucol® is non-toxic to cells.

MDCK cells were incubated with serial dilutions of Sambucol® for 4 hours. After this time, a crystal violet test was performed and cell survival measured by optical density. Data are expressed as percentage survival as compared to positive (cell only) controls.

FIG. 2: Sambucol® dramatically reduces NIBRG-14 titre.

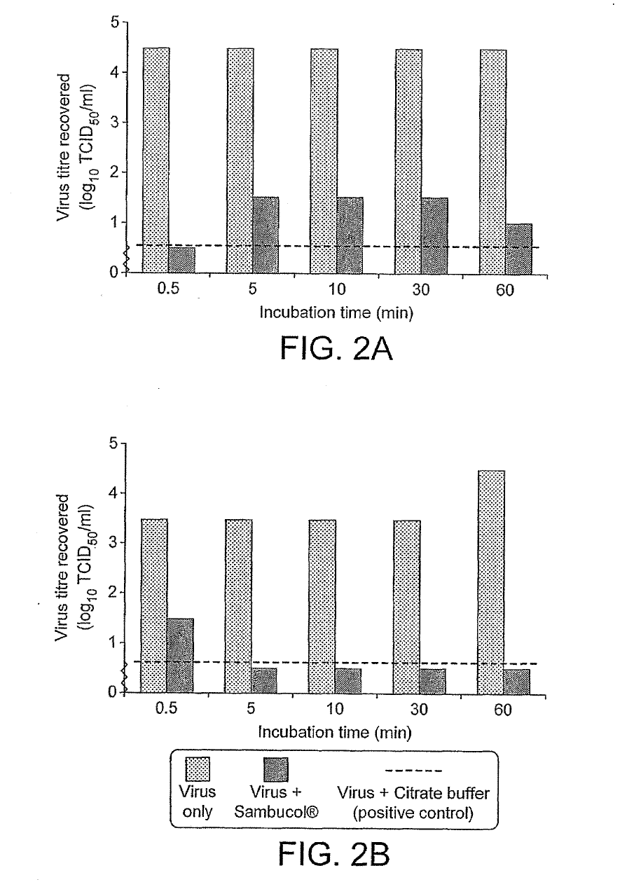

Avian influenza virus NIBRG-14 was incubated with a 1/4 (A) and 1/8 (B) dilutions of Sambucol®, or citrate buffer (antiviral control) for different lengths of time before virus was allowed to infect MDCK cells. 3 days later, viral titre was assessed. Graphs display results as −log₁₀ TCID₅₀/ml. The virus titre recovered from the citrate buffer antiviral control is overlaid. The lower detection limit if this assay is 0.5−log₁₀ TCID₅₀/ml, as indicated on y axis. TCID=tissue culture infectious dose.

### DETAILED DESCRIPTION OF THE INVENTION

The aim of this study was to determine the antiviral activity of Sambucol®, a standardised Black Elderberry extract, against avian influenza NIBRG-14 (H5N1) virus. Antiviral assays were performed in MDCK cells using two Sambucol® dilutions at several incubation times. Results show at least 99% reduction in the titre of avian influenza NIBRG-14 (H5N1), namely 2.0 log₁₀ TCID/ml. Therapeutic index calculations indicated direct influence of Sambucol® on titre reduction. Further studies evaluate the effectiveness of Sambucol® against avian influenza (H5N1) virus in animals and humans.

The results presented herein suggest that the elderberry extract (Sambucol®) has at least one of the following properties:
it is capable of inhibiting viral induced haemagglutination;
it is capable of inhibiting viral adsorption to a susceptible host cell;
it is capable of inhibiting viral penetration into susceptible host cell (e.g. by endocytosis);
it is capable of inhibiting viral replication, thereby preventing propagation of viral progenies.

Generally host cells infected by the influenza virus, to be treated with the elderberry extract of the invention are epithelial cells, or cells from the respiratory tract.

The isolated elderberry extract described in the invention is effective for treatment of a subject against a viral infection. According to the present invention the term “treatment” denotes any physiological effect resulting in the therapeutic treatment of a viral infection or prevention (prophylactic treatment) of the formation of a viral infection in a subject exposed to an infectious virus, particularly the avian influenza type A (H5N1) virus.

This, in the context of the present invention, treatment refers to at least one of the following; inhibition of viral adsorption to susceptible cells, inhibition of viral penetration into susceptible cells, inhibition of viral replication, all of which result in a decrease in viral load (thereby less antibody response) in an infected subject, preferably total elimination of the virus from the subject. Treatment preferably results in the improved and more rapid recovery of the subject from the viral infection or the prevention of the infection from occurring.

The effective amount of elderberry extract is determined by such consideration as may be known in the art. The amount must be effective to achieve the desired therapeutic or prophylactic effect described above. The amount is typically determined in appropriately designed studies and the person versed in the art will know how to properly conduct such studies in order to determine the required effective amount.

The viral infection according to the invention is an infection preferably localised within the respiratory tract, either the upper respiratory tract, lower respiratory tract, or both.

According to one embodiment, the virus causing the infection is a member of the Orthomyxoviridae family. According to a specific embodiment, the virus is selected from Influenza viruses, particularly the avian influenza virus type A (H5N1).

It is commonly known that viral infections, and especially influenza H5N1 viral infection, may be transmitted from animals to animals, animals to humans and it is likely transmitted from humans to humans and from humans to animals as well. Thus, the invention is also applicable for treating subjects where the virus is an animal-type virus being transmitted from an animal to humans, and vice versa. Thus, the subject to be treated by the elderberry extract of the invention, may be a human, or an animal, like duck, chicken, goose, turkey, pheasant, quail, dove, pigeon, ostrich, partridge, pig, cow, buffalo, sheep goat or horse.

The elderberry extract may be administered in various ways suitable for anti-viral therapy. It should be noted that it can be administered as the dry extract (e.g. as a powder), or within a suitable carrier suitable for a selected delivery route. The extract may be administered by any suitable route, e.g. orally, or via intranasal application, e.g. by inhalation.

The elderberry extract described herein is preferably intended for oral administration and can be in liquid forms, e.g. syrup or oral spray, as well as in solid forms, e.g. tablets, coated tablets, chewing tablets, multi-layered tablets and capsules. The various dosage forms can be designed for the sustained release of the active ingredient.

An example for a liquid aqueous formulation according to the invention is a syrup comprising the anti-virally active Sambucus nigra extract and as optional additives raspberry juice, citric acid, natural or synthetic sweetening and/or flavouring agents, such as honey, and conventional preservatives, such as p-hydroxybenzoic acid derivatives.

Solid formulations in tablet form may comprise, in addition to the said anti-viral active extract, additives such as sorbitol, xylitol and optionally vitamin C.

A specific formulation of the elderberry extract of the invention has the following composition: Glucose syrup, Sambucus nigra L. extract (38% w/w), raspberry extract, citric acid, natural flavours and preservatives.

According to another embodiment, the isolated extract is administered in the form of a solution, e.g. water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.0 or, from pH 6.0 to pH 7.0. Buffers should be physiologically compatible and include, simply by way of example, phosphate buffers. For example, a representative nasal decongestant is described as being buffered to a pH of about 6.2 [Remington's Pharmaceutical Sciences, Ed. By Arthur Osol, p 1445 (1980)]. Of course, the ordinary artisan can readily determine a suitable saline content and pH for an innocuous aqueous carrier for nasal administration.

Other, non-limiting examples of intranasal dosage from include nasal gels, creams, pastes or ointments with a viscosity of e.g. from about 10 to about 3000 cps, or from about 2500 to 6500 cps, or greater, which may provide a more sustained contact with the nasal mucosal surfaces. Such carrier viscous formulations may be based upon, simply by way of example, polymeric carriers such as alkylcelluloses and/or other biocompatible carriers of high viscosity well known to the art [see Remington's, cited supra]. The carrier containing the isolated fraction may also be soaked into a fabric material, such as gauze, that can be applied to the nasal mucosal surface to allow for active substances in the isolated fraction to penetrate to the mucosa.

Other ingredients, such as art known preservatives, colorants, lubricating or viscous mineral or vegetable oils, perfumes, natural or synthetic plant extracts such as aromatic oils, and humectants and viscosity enhancers such as e.g. glycerol, can also be included to provide additional viscosity, moisture retention and a pleasant texture and odour for the formulation.

Further, for nasal administration of solutions or suspensions of the isolated fraction, various devices are available in the art for the generation of drops, droplets and sprays. For example, solutions comprising the isolated fraction can be administered into the nasal passage by means of a simple dropper (or pipette) that includes a glass, plastic or metal dispensing tube from which the contents are expelled drop by drop by means of air pressure provided by a manually powered pump, e.g. a flexible rubber bulb, attached to one end. Fine droplets and sprays can be provided by a manual or electrically powered intranasal pump dispenser or squeeze bottle as well known to the art, e.g. that is designed to blow a mixture of air and fine droplets into the nasal passages.

The extract may be administered as is, or in the form of a composition comprising as active agent, the elderberry extract. Such a composition may further optionally comprise additional active agents that may be part of the treatment of each specific patient. Thus, for example, said composition may further optionally comprise antibiotics, vitamins, etc.

The preparation of pharmaceutical compositions and formulations is well know in the art and has been described in many articles and textbooks, see e.g. Remington's Pharmaceutical Sciences, Gennaro A. R. ed, Mack publishing Co., Eaton, Pa., 1990 and especially pp. 1521-1712 therein.

The present invention is defined in the claims, the contents of which are to read as included within the disclosure of the specification.

Disclosed and described, it is to be understood that this invention is not limited to the particular examples, process steps and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms of “a”, “an” and “the” include plurals referents unless the content clearly dictates otherwise.

Throughout this specification, and the claims which follow, unless the context requires otherwise, the word “comprise” and variations such as “comprises” and “comprising”, will be understood to imply the inclusion of a stated integer or step or group of integers or steps but not the exclusion of any other integer or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognise that numerous modifications can made without departing from the intended scope of the invention.

### EXAMPLES

### Experimental Procedures

The study was performed at the laboratories of Retroscreen Virology Ltd., St Bartholomew's & The Royal London School of Medicine & Dentistry, University of London.

All incubations were at 37° C., 5% CO₂ unless otherwise specified.

### Virus

The virus used was influenza NIBRG-14 (H5N1), characterised by A/PR/8 backbone, Ha and NA genes form an H5N1 isolate (A/Vietnam/1194/04) with lower pathogenicity (supplied by the National Institute for Biological Standards and Control).

### Sambucol® (Elderberry Extract)

Sambucol® was supplied as a sterile filtered undiluted preparation. The test dilutions were 1/4 and 1/8 of the original preparation. In the virucidal assay each dilution of Sambucol® underwent a 9/10 dilution upon mixing with the virus.

Specifically, Sambucol® syrup typically contains (in g/100 ml):
glucose syrup (85)
Elderberry extract (37.5)
Raspberry extract (1)
Citric acid (0.5)
Natural flavour (1)
Preservatives.

Similarly to the Sambucol® tablet described below, low calorie syrup contains liquid sorbitol instead of glucose syrup.

A Sambucol® tablet typically contains:
Black elderberry extract (freeze dried)
Sorbitol
Vitamin C

### Target Cells

Madin-Darby Canine Kidney Epithelial cells (MDCK) were used. 100 μl cell suspension at 5×10⁴ cells/ml was seeded onto each well of a 96-well plates and incubated for 24 h. Prior to use in the assays, plates were washed twice with PBS (100 μl/well) and 100 μl standard MDCK infection media was added to each well.

Preparation of Sambucus nigra L Extract

Sambucus nigra L extract (Sambucol®) was prepared as described previously [U.S. Pat. No. 4,742,046]. Briefly, fruits of Sambucus nigra (also known as Elderberry) are pressed without crushing the seeds and the extract is recovered by centrifugation and filtration, followed by ultra-centrifugation.

### Example 1

### Cytotoxicity

### Crystal Violet Assay

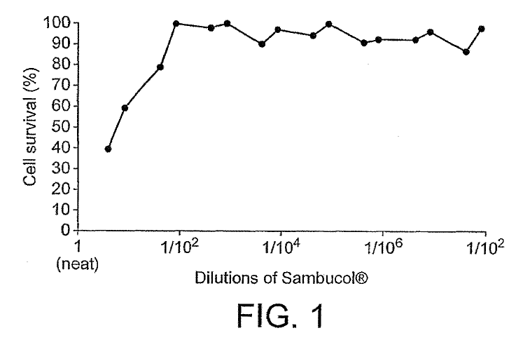

The crystal violet assay performed was used to calculate the viability of MDCK cell after incubation with various concentration of Sambucol®. The results are shown in FIG. 1. Dilution of Sambucol® to 1:80 or more completely eliminated any toxic effect of Sambucol® on MDCK cell.

### Cell Observation

The survivability threshold for toxicity in this assay was considered to be 90%.

### Example 2

### Reduction of Viral Titre Assay

40 μl of virus (total titre in reaction 4.0−log₁₀ TCID₅₀/ml) was added to 360 μl test sample and incubated at room temperature for 0.5, 5, 10, 30 and 60 minutes. The reaction mixture was added to the target cells and titrated across the 96-well plate following a 10-fold dilution series. The plate was then incubated for 60 minutes. Supernatants were then discarded and plates washed twice with PBS before 100 μl standard infection media was added to each well. Cells were incubated for 3 days at which point viral titre was measured.

The antiviral positive control consisted of a 5 minute pre-treatment of virus with citrate buffer at pH 3.5 (known incubation time point of citrate buffer that exhibits antiviral activity against influenza A viruses—unpublished data).

### Therapeutic Index

The therapeutic index is an indication of the specificity of the toxicity of a substance to the virus, as opposed to the host cells, and it is expressed as a ratio of the reduction in viral titre to the reduction in cell viability. The calculations show that the reduction in viral titre is directly due to the presence of Sambucol®.

Table 1 shows the analysis of the data from the reduction of viral titre assay. As may be depicted from the values presented in the table, Sambucol® reduced the viral titre recovered from infected MDCK cells by over 99% at both concentrations and all time points tested.

TABLE 1
Sambucol ® is over 99% effective at reducing viral titre

| Time of | | | | |
|---|---|---|---|---|
| incubation | Sambucol ® ¼ dilution | | Sambucol ® ⅛ dilution | |
| (min) | (−log₁₀TCID₅₀/ml) | (%) | (−log₁₀TCID₅₀/ml) | (%) |
| 0.5 | >4.0 | >99.90 | 2..0 | 99.0 |
| 5 | 3.0 | 99.90 | >3.0 | >99.90 |
| 10 | 3.0 | 99.90 | >3.0 | >99.90 |
| 30 | 3.0 | 99.90 | >3.0 | >99.90 |
| 60 | 3.5 | 99.97 | >4.0 | >99.90 |

This study aimed to determine the antiviral activity of Sambucol® against the avian NIBRG-14 (H5N1) influenza virus. Both 1/4 and 1/8 dilutions were at least 99% effective and reduced the titre of avian influenza NIBRG-14 (H5N1) by at least 2.0−log₁₀ TCID₅₀/ml. A reduction of 1−log₁₀ TCID₅₀/ml has previously been deemed significant (Oxford, J. S. et al. (1994) Sodium deoxycholate exerts a direct destructive effect on HIV and influenza viruses in vitro and inhibits retrovirus-induced pathology in an animal model. J. Antimicrob Chemother 45(5):617-621]. Sambucol® was therefore significantly effective at reducing the titre of avian influenza NIBRG-14 (H5N1) virus. Sambucol® was as effective as citrate buffer positive control at reducing viral titre. The therapeutic index calculated for both dilutions of Sambucol® indicate that the reduction in titre was due to the action of Sambucol® on the avian influenza NIBRG-14 virus.

Sambucol®, which has previously been shown to be effective against human influenza viruses A and B in vitro and in vivo, has now been demonstrated to have anti-viral properties against avian influenza H5N1 virus.

### Example 3

### In Vivo Administration of Sambucol® for H5N1 Influenza Virus Treatment

Four groups of mice aged 4 to 6 weeks are used for the experiment. Group I is administered influenza type A virus (H5N1). Group II is administered influenza type A virus (H5N1) previously incubated with Sambucol®. Group III is administered Sambucol® only. Group IV is the control group to which no virus or Sambucol® is given.

Preliminary results show that no mortality was observed in Groups III and IV, reassuring that the formulation of the invention is not toxic to the animals.

1. The use of a Sambucus nigra L. extract in the preparation of a pharmaceutical formulation for the treatment of an influenza viral infection in a subject in need thereof.

2. The use as claimed in claim 1, wherein the influenza viral infection is caused by influenza type A virus H5N1.

3. The use as claimed in claim 1, wherein the subject in need is a human.

4. The use as claimed in claim 1, wherein the Sambucus nigra L extract is SAMBUCOL®.

5. A method of treating an influenza viral infection comprising administering a therapeutically effective amount of a Sambucus nigra L. extract to a subject in need thereof.

6. The method as claimed in claim 5, wherein the influenza viral infection is caused by the influenza type A virus H5N1.

7. The method as claimed in claim 5, wherein the subject in need is human.

8. The method as claimed in claim 5, wherein the Sambucus nigra L. extract is SAMBUCOL®.

9. A method of inhibiting influenza viral infection comprising contacting a cell infected with the influenza virus with an effective amount of Sambucus nigra L extract.

10. The method of claim 9, wherein the influenza virus is the influenza type A virus H5N1.
